(19) United States
(12) Patent Application Publication
(10) Pub. No.: US 2005/0240037 A1
(43) Pub. Date: Oct. 27, 2005

(54) Method for the continuous intermediate separation of the solvent used in the oxirane synthesis with no coupling product

(75) Inventors: Peter Bassler, Viernheim (DE); Hans-Georg Gobbel, Kallstadt (DE); Joaquin Henrique Teles, Otterstadt (DE); Peter Rudolf, Ladenburg (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/521,359

(22) PCT Filed: Jul. 22, 2003
(86) PCT No.: PCT/EP03/07990

§ 371 Date: Jan. 14, 2005

(30) Foreign Application Priority Data
Jul. 23, 2002 (DE) 102 33 381.5

(51) Int. Cl. C07D 301/19

(52) U.S. Cl. 549/529

(57) ABSTRACT

Process for the continuously operated distillation of the solvent used in the synthesis of an oxirane by reaction of a hydroperoxide with an organic compound, wherein the mixture comprising the solvent which is obtained in the synthesis and subsequent work-up is separated in a dividing wall column into a low-boiling fraction, an intermediate-boiling fraction and a high-boiling fraction and the solvent is taken off as intermediate-boiling fraction from the side offtake of the column.

The present invention relates to a process for the continuously operated distillation of the solvent used in the synthesis of oxiranes with the low boilers and high boilers being separated off simultaneously, wherein the mixture comprising the solvent is fractionated in a dividing wall column having a side offtake and the solvent is obtained as intermediate-boiling fraction from the side offtake. In a particular embodiment, the dividing wall column can also be in the form of two thermally coupled columns. The oxiranes are preferably prepared without formation of coproducts by reaction of a hydroperoxide with a suitable organic compound.

In customary processes of the prior art, oxiranes can be prepared by reaction of suitable organic compounds with hydroperoxides in single-stage or multistage reactions.

For example, the multistage process described in WO 00/07965 provides for the reaction of the organic compound with a hydroperoxide to comprise at least the steps (i) to (iii):
(i) reaction of the hydroperoxide with the organic compound to give a product mixture comprising the reacted organic compound and unreacted hydroperoxide,
(ii) separation of the unreacted hydroperoxide from the mixture resulting from step (i),
(iii) reaction of the hydroperoxide which has been separated off in step (ii) with the organic compound.

Accordingly, the reaction of the organic compound with the hydroperoxide takes place in at least two steps (i) and (iii), with the hydroperoxide separated off in step (ii) being reused in the reaction.

The reactions in steps (i) and (iii) are preferably carried out in two separate reactors, preferably fixed-bed reactors, with the reaction of step (i) preferably taking place in an isothermal reactor and the reaction of step (iii) taking place in an adiabatic reactor.

In this sequence, preference is given to using hydrogen peroxide as hydroperoxide, bringing the organic compound into contact with a heterogeneous catalyst during the reaction and carrying out the reaction in a solvent. In particular, alkenes can be reacted as organic compound.

Owing to the high selectivity of the reaction, this method of preparation is also referred to as coproduct-free oxirane synthesis.

The above process can be used especially for preparing propylene oxide from propylene and hydrogen peroxide as oxidant. Here, the hydrogen peroxide conversion in step (i) is from about 85% to 90% and that in step (iii) is about 95% based on the second step. Over both steps, a total hydrogen peroxide conversion of about 99% can be achieved at a propylene oxide selectivity of about 94-95%.

If the reaction is carried out in methanol as solvent, the propylene oxide formed has to be separated from a mixture which further comprises, for example, methanol as solvent, water, by-products such as methoxypropanols, 1,2-propylene diglycol, acetaldehyde, methyl formate, unreacted propylene as organic compound and hydrogen peroxide as hydroperoxide. The propylene oxide is isolated from this mixture by distillation.

Thus, the work-up of the oxirane, for example propylene oxide, by distillation always gives streams comprising the solvent together with further impurities.

The separation processes carried out in the past to recover the solvent, for instance to enable it to be reused for the oxirane synthesis, have hitherto typically been carried out in distillation columns having a side offtake or in columns connected in series. This procedure requires an increased outlay in terms of energy and apparatus.

It is an object of the present invention to optimize the distillation of the solvent used in the preferably coproduct-free oxirane synthesis by reaction of a hydroperoxide with an organic compound so as to reduce the otherwise customary energy consumption. The solvent should be obtained in a quality which enables it to be reused for the oxirane synthesis in question.

We have found that this object is achieved by a continuously operated process for the distillation in a dividing wall column of the solvent used in the preferably coproduct-free synthesis of an oxirane by reaction of a hydroperoxide with an organic compound.

The present invention accordingly provides a continuously operated process for the distillation of the solvent used in the synthesis of an oxirane by reaction of a hydroperoxide with an organic compound, wherein the mixture comprising the solvent which is obtained in the synthesis and subsequent work-up is separated in a dividing wall column into a low-boiling fraction, an intermediate-boiling fraction and a high-boiling fraction and the solvent is taken off as intermediate-boiling fraction from the side offtake of the column.

The process of the present invention enables the solvent to be obtained in good purity, while the energy consumption can be reduced compared to the distillation methods used hitherto. The solvent can thus also be reused, for example, for the oxirane synthesis. Compared to the processes disclosed in the prior art, the new process of the present invention leads to a reduced layout in terms of apparatus. Furthermore, the dividing wall column has a particularly low energy consumption and thus offers advantages in terms of the energy requirement over a conventional column or an assembly of conventional columns. This is highly advantageous for industrial use.

Distillation columns having side offtakes and a dividing wall, hereinafter also referred to as dividing wall columns, are known. They represent a further development of distillation columns which have only a side offtake but no dividing wall. The use of the last-named type of column is restricted because the products taken off at the side offtakes are never completely pure. In the case of products taken off at the side offtakes in the reinforcement section of the column, which are usually taken off in liquid form, the side product still contains proportions of low-boiling components which should be separated off via the top. In the case of products taken off at side offtakes in the stripping section of the column, which are usually taken off in gaseous form, the side product still contains proportions of high boilers. The use of conventional side offtake columns is therefore restricted to cases in which contaminated side products are permissible.

However, when a dividing wall is installed in such a column, the separation action can be improved. This type of construction makes it possible for side products to be taken off in pure form. A dividing wall is installed in the middle region above and below the feed point and the side offtake. This can be fixed in place by welding or can be merely pushed into place. It seals off the offtake section from the inflow section and prevents crossmixing of liquid and vapor streams over the entire column cross section in this part of the column. This reduces the total number of distillation columns required in the fractionation of multicomponent mixtures whose components have similar boiling points.

This type of column has been used, for example, for the separation of an initial mixture of the components methane, ethane, propane and butane (U.S. Pat. No. 2,471,134), for the separation of a mixture of benzene, toluene and xylene (U.S. Pat. No. 4,230,533) and for the separation of a mixture of n-hexane, n-heptane and n-octane (EP 0 122 367).

Dividing wall columns can also be used successfully for separating mixtures which boil azeotropically (EP 0 133 510).

Finally, dividing wall columns in which chemical reactions can be carried out with simultaneous distillation of the products are also known. Examples which may be mentioned are esterifications, transesterifications, saponifications and acetalizations (EP 0 126 288).

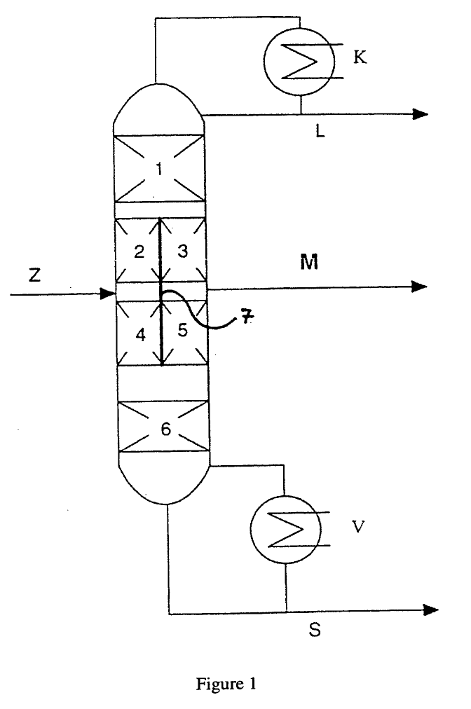

FIG. 1 schematically shows the distillation of the solvent used in the oxirane synthesis in a dividing wall column having a side offtake. Here, the solvent mixture from the oxirane synthesis is continuously introduced as feed Z into the dividing wall column. In the column, this mixture is separated into a fraction comprising the low boilers L, an intermediate-boiling fraction comprising the solvent and a fraction comprising the high boilers S.

At the side offtake for intermediate boilers M, the solvent as material of value is taken off in liquid or gaseous form. To take off this fraction at the side offtake, it is possible to use receivers in which the liquid or condensing vapor can be collected and which may be located either inside or outside the column.

Such a dividing wall column preferably has from 15 to 60, more preferably from 20 to 35, theoretical plates. The process of the present invention can be carried out particularly advantageously using such a design.

Accordingly, the dividing wall column in a preferred embodiment of the process of the present invention has from 15 to 60 theoretical plates.

The upper, combined region 1 of the inflow and offtake part of the dividing wall column preferably has from 5 to 50%, more preferably from 15 to 30%, of the total number of theoretical plates in the column, the enrichment section 2 of the inflow part preferably has from 5 to 50%, more preferably from 15 to 30%, the stripping section 4 of the inflow part preferably has from 5 to 50%, more preferably from 15 to 30%, the stripping section 3 of the offtake part preferably has from 5 to 50%, more preferably from 15 to 30%, the enrichment section 5 of the offtake part preferably has from 5 to 50%, more preferably from 15 to 30%, and the combined lower region 6 of the inflow and offtake part of the dividing wall column preferably has from 5 to 50%, more preferably from 15 to 30%, in each case of the total number of theoretical plates in the column. The dividing wall 7 prevents mixing of liquid and vapor streams.

The sum of the number of theoretical plates in the regions 2 and 4 in the inflow part is preferably from 80 to 110%, more preferably from 90 to 100%, of the sum of the number of theoretical plates in the regions 3 and 5 in the offtake part.

It is likewise advantageous for the feed point and the side offtake to be arranged at different heights in the column relative to the position of the theoretical plates. The feed point is preferably located from one to eight, more preferably from three to five, theoretical plates above or below the side offtake.

The dividing wall column used in the process of the present invention is preferably configured either as a packed column containing random packing or ordered packing or as a tray column. For example, it is possible to use sheet metal or mesh packing having a specific surface area of from 100 to 1000 m²/m³, preferably from about 250 to 750 m²/m³, as ordered packing. Such packing provides a high separation efficiency combined with a low pressure drop per theoretical plate.

In the abovementioned configuration of the column, the region of the column divided by the dividing wall, which consists of the enrichment section 2 of the inflow part, the stripping section 3 of the offtake part, the stripping section 4 of the inflow part and the enrichment section 5, or parts thereof is/are preferably provided with ordered packing or random packing, and the dividing wall 7 is preferably thermally insulated in these regions.

The solvent mixture to be fractionated is introduced continuously into the column in the form of the feed stream Z comprising the low boilers, intermediate boilers and high boilers. This feed stream is generally liquid. However, it can be advantageous to subject the feed stream to preliminary vaporization and subsequently introduce it into the column as a two-phase, i.e. gaseous and liquid, mixture or in the form of one gaseous stream and one liquid stream. This preliminary vaporization is particularly useful when the feed stream contains relatively large amounts of low boilers. The preliminary vaporization enables a considerable load to be taken off the stripping section of the column.

The feed stream is advantageously metered by means of a pump or via a static inflow head of at least 1 m into the inflow part. This inflow is preferably regulated via a cascade regulation in combination with the regulation of the liquid level in the inflow part. The regulation is set so that the amount of liquid introduced into the enrichment section 2 cannot drop below 30% of the normal value. It has been found that such a procedure is important to even out troublesome fluctuations in the amount or concentration of the feed.

It is likewise important for the division of the liquid flowing down from the stripping section 3 of the offtake part of the column between the side offtake and the enrichment section 5 of the offtake part is set by means of a regulation device so that the amount of liquid going to the region 5 cannot drop below 30% of the normal value.

Adherence to these prerequisites has to be ensured by means of appropriate regulation methods.

Regulation mechanisms for the operation of dividing wall columns have been described, for example, in Chem. Eng. Technol. 10 (1987) 92-98, Chem.-Ing.-Technol. 61 (1989), No. 1, 16-25, Gas Separation and Purification 4 (1990) 109-114, Process Engineering 2 (1993) 33-34, Trans IChemE 72 (1994) Part A 639-644, Chemical Engineering 7 (1997) 72-76. The regulation mechanisms described in this prior art can also be employed for or applied to the process of the present invention.

The regulation principle described below has been found to be particularly useful for the continuously operated distillation of the solvent. It is readily able to cope with fluctuations in loading. The distillate is thus preferably taken off under temperature control.

A temperature regulation device which utilizes the downflow quantity, the reflux ratio or preferably the quantity of runback as regulating parameter is provided in the upper section 1 of the column. The measurement point for the temperature regulation is preferably located from three to eight, more preferably from four to six, theoretical plates below the upper end of the column.

Appropriate setting of the temperature then results in the liquid flowing down from the section 1 of the column being divided at the upper end of the dividing wall so that the ratio of the liquid flowing to the inflow part to that flowing to the offtake part is preferably from 0.1 to 1.0, more preferably from 0.3 to 0.6.

In this method, the downflowing liquid is preferably collected in a receiver which is located in or outside the column and from which the liquid is then fed continuously into the column. This receiver can thus take on the task of a pump reservoir or provide a sufficiently high static head of liquid which makes it possible for the liquid to be passed on further in a regulated manner by means of regulating devices, for example valves. When packed columns are used, the liquid is firstly collected in collectors and from there conveyed to an internal or external receiver.

The vapor stream at the lower end of the dividing wall is set by selection and/or dimensioning of the separation internals and/or incorporation of pressure-reducing devices, for example orifice plates, so that the ratio of the vapor stream in the inflow part to that in the offtake part is preferably from 0.8 to 1.2, preferably from 0.9 to 1.1.

In the abovementioned regulation principle, a temperature regulation device which utilizes the quantity taken off at the bottom as regulating parameter is provided in the lower combined section 6 of the column. The bottom product can therefore be taken off under temperature control. The measurement point for the temperature regulation device is preferably located from three to six, more preferably from four to six, theoretical plates above the lower end of the column.

In addition, the level regulation in column section 6 (bottom of the column) can be utilized for regulating the quantity taken off at the side offtake. For this purpose, the liquid level in the vaporizer is used as regulating parameter.

The differential pressure over the column can also be utilized as regulating parameter for the heating power. The distillation is advantageously carried out at a pressure of from 0.5 to 15 bar, preferably from 5 to 13 bar. The pressure is measured at the top of the column. Accordingly, the heating power of the vaporizer at the bottom of the column is selected to maintain this pressure range.

This results in a distillation temperature of preferably from 30 to 140° C., more preferably from 60 to 140° C., in particular from 100 to 130° C. The distillation temperature is measured at the side offtake.

In a preferred embodiment of the process of the present invention, the pressure in the distillation is therefore from 0.5 to 15 bar and the distillation temperature is from 30 to 140° C.

To be able to operate the dividing wall column in a trouble-free manner, the abovementioned regulation mechanisms are usually employed in combination.

In the separation of multicomponent mixtures into low-boiling, intermediate-boiling and high-boiling fractions, there are usually specifications in respect of the maximum permissible proportion of low boilers and high boilers in the middle fraction. Here, individual components which are critical to the separation problem, referred to as key components, or else the sum of a plurality of key components are/is specified.

Adherence to the specification for the high boilers in the intermediate-boiling fraction is preferably regulated via the division ratio of the liquid at the upper end of the dividing wall. The division ratio is set so that the concentration of key components for the high-boiling fraction in the liquid at the upper end of the dividing wall amounts to from 10 to 80% by weight, preferably from 30 to 50% by weight, of the value which is to be achieved in the stream taken off at the side. The liquid division can then be set so that when the concentration of key components of the high-boiling fraction is higher, more liquid is introduced into the inflow section, and when the concentration of key components is lower, less liquid is introduced into the inflow section.

Accordingly, the specification for the low boilers in the intermediate-boiling fraction is regulated by means of the heating power. Here, the heating power in the vaporizer is set so that the concentration of key components for the low-boiling fraction in the liquid at the lower end of the dividing wall amounts to from 10 to 80% by weight, preferably from 30 to 50% by weight, of the value which is to be achieved in the product taken off at the side. Thus, the heating power is set so that when the concentration of key components of the low-boiling fraction is higher, the heating power is increased, and when the concentration of key components of the low-boiling fraction is lower, the heating power is reduced.

The concentration of low and high boilers in the intermediate-boiling fraction can be determined by customary analytical methods. For example, infrared spectroscopy can be used for detection, with the compounds present in the reaction mixture being identified by means of their characteristic absorptions. These measurements can be carried out in-line directly in the column. However, preference is given to using gas-chromatographic methods. In this case, sampling facilities are then provided at the upper and lower end of the dividing wall. Liquid or gaseous samples can then be taken continuously or at intervals from the column and analyzed to determine their compositions. The appropriate regulation mechanisms can then be activated as a function of the composition.

An objective of the process of the present invention is to make the solvent available in a purity of preferably at least 95%. The concentration of key components of the low boilers and of key components of the high boilers in the solvent should then preferably be less than 5% by weight, based on a total of solvent and key components of 100% by weight.

When methanol is used as solvent, low-boiling key components are, for example, acetaldehyde and methyl formate and high-boiling key components are, for example, methoxypropanols, propylene glycol and water.

In a specific embodiment of the dividing wall column, it is also possible for the inflow part and the offtake part which are separated from one another by the dividing wall 7 not to be present in one column but to be physically separate from one another. In this specific embodiment, the dividing wall column can thus comprise at least two physically separate columns which then have to be thermally coupled with one another.

Accordingly, a particular embodiment of the process of the present invention provides for the dividing wall column to be in the form of two thermally coupled columns.

Such thermally coupled columns generally exchange both vapor and liquid between them. However, in specific embodiments, it is also possible for them to exchange only liquid between them. This specific embodiment then has the advantage that the thermally coupled columns can also be operated under different pressures, which can make it possible to achieve better setting of the temperature level required for the distillation than in the case of a conventional dividing wall column. Often only one of the thermally coupled columns is equipped with a vaporizer.

Such thermally coupled columns are usually operated in such a way that the low-boiling fraction and the high-boiling fraction are taken off from different columns. The operating pressure of the column from which the low-boiling fraction is taken is preferably from about 0.5 to 3 bar higher than the pressure in the column from which the high-boiling fraction is taken.

In the case of coupled columns, it can also be advantageous to vaporize bottom streams either completely or partly in an additional vaporizer and only then feed them to the next column. This prevaporization is particularly useful when the bottom stream from the first column contains relatively large amounts of intermediate boilers. In this case, the prevaporization can be carried out at a lower temperature and the load on the vaporizer of the second column can be reduced, if this column is equipped with a vaporizer.

Furthermore, this measure significantly decreases the load on the stripping section of the second column. The prevaporized stream can be fed to the subsequent column either as a two-phase stream or in the form of two separate streams.

Conversely, it is, however, also possible for low-boiling streams taken from the top to be condensed either partly or completely before they are fed to the next column. This measure can also contribute to better separation between low boilers and intermediate boilers present therein.

In further embodiments of the process of the present invention, the liquid bottom stream taken from one of the coupled columns is then partly or completely vaporized before it is fed to the other column, and/or the gaseous top stream taken from one of the coupled columns is partly or completely condensed before it is fed to the other column.

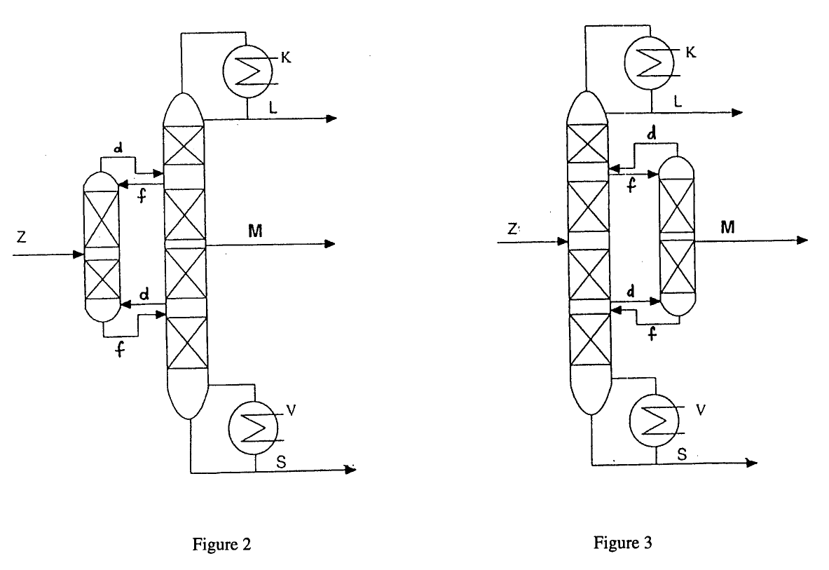

Examples of dividing wall columns in the specific embodiment of thermally coupled columns are shown schematically in FIGS. 2, 3, 4 and 5. These arrangements comprising two coupled columns are preferably employed when an intermediate boiler and a high-boiling fraction and a low-boiling fraction are to be separated off simultaneously from an intermediate-boiling fraction. These arrangements thus represent specific variants of a dividing wall column having a side offtake.

The methanol used as solvent in the synthesis of propylene oxide can advantageously be separated off as intermediate boiler M from acetaldehyde and methyl formate as low boilers L and methoxypropanols, propylene glycol and water as high boilers S.

FIG. 2 shows two thermally coupled columns where the column into which the feed Z is introduced exchanges vapor d and liquid f with the downstream column both via the top and via the bottom. The energy is introduced essentially via the vaporizer V of the column located downstream of the feed column. Here, the low boilers L can be obtained via the top of the downstream column by condensation in the condenser K, the intermediate boilers M can be obtained from the side offtake and the high boilers S can be obtained from the bottom.

An arrangement as outlined in FIG. 3 is also possible. Here, the low boilers L can be separated off at the top and the low boilers S can be separated off at the bottom in the feed column. The intermediate boilers M are obtained from the side offtake of the downstream column. The downstream column can exchange vapor d and liquid f with the feed column via both the top and the bottom. Energy is introduced essentially via the vaporizer of the feed column.

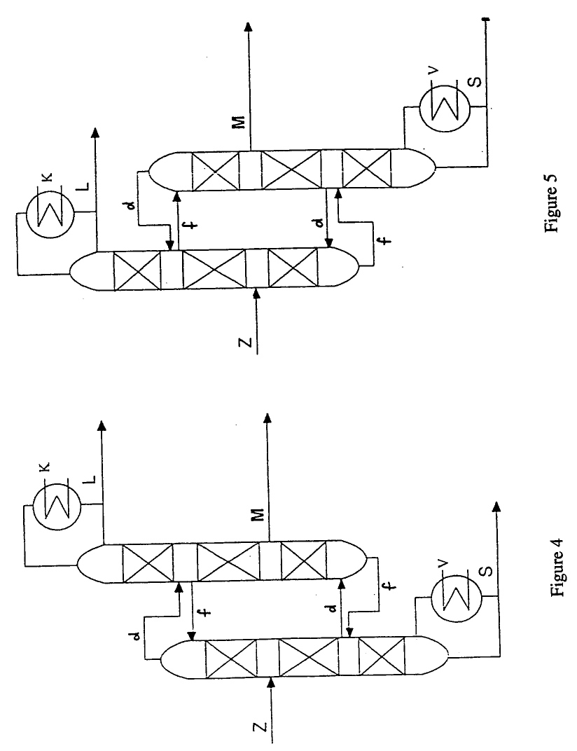

FIG. 4 shows an arrangement in which the high boilers S are obtained in the bottoms of the feed column. The low boilers L are obtained at the top of the downstream column and the intermediate boilers M are obtained via the side offtake of the downstream column. Energy is introduced essentially via the vaporizer of the feed column.

FIG. 5 shows an arrangement in which the low boilers L are obtained via the top of the feed column. In the downstream column, the high boilers S are obtained as bottoms and the intermediate boilers M are obtained via the side offtake. Energy is introduced essentially via the vaporizer of the column located downstream of the feed column.

In an embodiment of the process of the present invention, therefore, the solvent mixture is separated into the low-boiling, intermediate-boiling and high-boiling fractions in the column located downstream of the feed column, or
the low-boiling and high-boiling fractions are taken off from the solvent mixture in the feed column and the intermediate-boiling fraction is taken off in the downstream column, or
the high-boiling fraction is taken off from the solvent mixture in the feed column and the low-boiling and intermediate-boiling fractions are taken off in the downstream column, or
the low-boiling fraction is taken off from the solvent mixture in the feed column and the intermediate-boiling and high-boiling fractions are taken off in the downstream column.

The columns of FIGS. 2 to 5 can also be configured as packed columns containing random packing or ordered packing or as tray columns. For example, sheet metal or mesh packing having a specific surface area of from 100 to 1000 m²/m³, preferably from about 250 to 750 m²/m³, can be used as ordered packing. Such packing provides a high separation efficiency combined with a low pressure drop per theoretical plate.

The oxirane synthesis providing the feed to the process of the present invention for the continuously operated distillation of the solvent used in the preferably coproduct-free oxirane synthesis can be carried out using the starting materials known from the prior art.

Preference is given to using organic compounds which have at least one C—C double bond. Examples of such organic compounds having at least one C—C double bond include the following alkenes:
ethene, propylene, 1-butene, 2-butene, isobutene, butadiene, pentenes, piperylene, hexenes, hexadienes, heptenes, octenes, diisobutene, trimethylpentene, nonenes, dodecene, tridecene, tetradecene to eicosene, tripropene and tetrapropene, polybutadienes, polyisobutenes, isoprene, terpenes, geraniol, linalool, linalyl acetate, methylenecyclopropane, cyclopentene, cyclohexene, norbornene, cycloheptene, vinylcyclohexane, vinyloxirane, vinylcyclohexene, styrene, cyclooctene, cyclooctadiene, vinylnorbornene, indene, tetrahydroindene, methylstyrene, dicyclopentadiene, divinylbenzene, cyclododecene, cyclododecatriene, stilbene, diphenylbutadiene, vitamin A, beta-carotene, vinylidene fluoride, allyl halides, crotyl chloride, methallyl chloride, dichlorobutene, allyl alcohol, methallyl alcohol, butenols, butenediols, cyclopentenediols, pentenols, octadienols, tridecenols, unsaturated steroids, ethoxyethene, isoeugenol, anethole, unsaturated carboxylic acids such as acrylic acid, methacrylic acid, crotonic acid, maleic acid, vinylacetic acid, unsaturated fatty acids such as oleic acid, linoleic acid, palmitic acid, naturally occurring fats and oils.

Preference is given to using alkenes containing from 2 to 8 carbon atoms. Particular preference is given to reacting ethene, propene and butene. Very particular preference is given to reacting propylene.

Propylene can be used as “chemical grade” propylene. It is then present together with propane in a volume ratio of propylene to propane of from about 97:3 to 95:5.

As hydroperoxides, it is possible to use the known hydroperoxides which are suitable for the reaction with the organic compound. Examples of such hydroperoxides are tert-butyl hydroperoxide and ethylbenzene hydroperoxide. Preference is given to using hydrogen peroxide as hydroperoxide for the oxirane synthesis, with an aqueous hydrogen peroxide solution also being able to be used.

The preparation of hydrogen peroxide can be carried out using, for example, the anthraquinone process by means of which virtually the entire world production of hydrogen peroxide is produced. This process is based on the catalytic hydrogenation of an anthraquinone compound to form the corresponding anthrahydroquinone compound, subsequent reaction of this with oxygen to form hydrogen peroxide and subsequent extraction to separate off the hydrogen peroxide formed. The catalysis cycle is closed by renewed hydrogenation of the anthraquinone compound which is obtained back.

An overview of the anthraquinone process is given in “Ullmann's Encyclopedia of Industrial Chemistry”, 5^{th} Edition, Volume 13, pages 447 to 456.

It is likewise conceivable to obtain hydrogen peroxide by converting sulfuric acid into peroxodisulfuric acid by anodic oxidation with simultaneous evolution of hydrogen at the cathode. Hydrolysis of the peroxodisulfuric acid then leads via peroxomonosulfuric acid to hydrogen peroxide and sulfuric acid, which is thus recovered.

It is of course also possible to prepare hydrogen peroxide from the elements.

In the synthesis of the oxirane from the hydroperoxide and the organic compound, one or more suitable catalysts can be added to increase the efficiency of the reaction. Here, heterogeneous catalysts are preferably used.

All heterogeneous catalysts which are suitable for the respective reaction are conceivable. Preference is given to using catalysts which comprise a porous oxidic material, e.g. a zeolite. The catalysts used preferably comprise a titanium-, germanium-, tellurium-, vanadium-, chromium-, niobium- or zirconium-containing zeolite as porous oxidic material.

Specific mention may be made of titanium-, germanium-, tellurium-, vanadium-, chromium-, niobium- and zirconium-containing zeolites having a pentasil zeolite structure, in particular the types which can be assigned X-ray-crystallographically to the ABW, ACO, AEI, AEL, AEN, AET, AFG, AFI, AFN, AFO, AFR, AFS, AFT, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CHI, CLO, CON, CZP, DAC, DDR, DFO, DFT, DOH, DON, EAB, EDI, EMT, EPI, ERI, ESV, EUO, FAU, FER, GIS, GME, GOO, HEU, IFR, ISV, ITE, JBW, KFI, LAU, LEV, LIO, LOS, LOV, LTA, LTL, LTN, MAZ, MEI, MEL, MEP, MER, MFI, MFS, MON, MOR, MSO, MTF, MTN, MTT, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG, ZON structure or to mixed structures comprising two or more of the abovementioned structures. Furthermore, titanium-containing zeolites having the ITQ4, SSZ-24, TTM-1, UTD-1, CIT-1 or CIT-5 structure are also conceivable for use in the process of the present invention. Further titanium-containing zeolites which may be mentioned are those of the ZSM-48 or ZSM-12 structure.

Particular preference is given to Ti zeolites having an MFI or MEL structure or an MFI/MEL mixed structure. Very particular preference is given to the titanium-containing zeolite catalysts which are generally referred to as “TS-1”, “TS-2”, “TS-3” and also Ti zeolites having a framework structure isomorphous with β-zeolite.

In particular, it is advantageous to use a heterogeneous catalyst comprising the titanium-containing silicalite TS-1.

It is possible to use the porous oxidic material itself as catalyst. However, it is of course also possible for the catalyst used to be a shaped body comprising the porous oxidic material. All processes known from the prior art can be used for producing the shaped body from the porous oxidic material.

Noble metals in the form of suitable noble metal components, for example in the form of water-soluble salts, can be applied to the catalyst material before, during or after the one or more shaping steps in these processes. This method is preferably employed for producing oxidation catalysts based on titanium silicates or vanadium silicates having a zeolite structure, and it is thus possible to obtain catalysts which contain from 0.01 to 30% by weight of one or more noble metals from the group consisting of ruthenium, rhodium, palladium, osmium, iridium, platinum, rhenium, gold and silver. Such catalysts are described, for example, in DE-A 196 23 609.6.

Of course, the shaped bodies can be processed further. All methods of comminution are conceivable, for example splitting or crushing the shaped bodies, as are further chemical treatments as are described above by way of example.

When a shaped body or a plurality thereof is used as catalyst, it/they can, after deactivation has occurred in the process of the present invention, be regenerated by a method in which the deposits responsible for deactivation are burned off in a targeted manner. This is preferably carried out in an inert gas atmosphere containing precisely defined amounts of oxygen-donating substances. This regeneration process is described in DE-A 197 23 949.8. It is also possible to use the regeneration processes mentioned there in the discussion of the prior art.

As solvents, it is possible to use all solvents which completely or at least partly dissolve the starting materials used in the oxirane synthesis. For example, it is possible to use water; alcohols, preferably lower alcohols, more preferably alcohols having less than six carbon atoms, for example ethanol, methanol, propanols, butanols, pentanols, diols or polyols, preferably those having less than 6 carbon atoms; ethers such as diethyl ether, tetrahydrofuran, dioxane, 1,2-diethoxyethane, 2-methoxyethanol; esters such as methyl acetate or butyrolactone; amides such as dimethylformamide, dimethylacetamide, N-methylpyrrolidone; ketones such as acetone; nitriles such as acetonitrile; sulfoxides such as dimethyl sulfoxide; aliphatic, cycloaliphatic and aromatic hydrocarbons, or mixtures of two or more of the abovementioned compounds.

Preference is given to using alcohols. The use of methanol as solvent is particularly preferred.

As reactors for the oxirane synthesis, it is of course possible to use all conceivable reactors which are best suited to the respective reactions. A reactor is not restricted to an individual vessel for the oxirane synthesis. Rather, it is also possible to use, for example, a cascade of stirred vessels.

Fixed-bed reactors are preferably used as reactors for the oxirane synthesis. Further preference is given to using fixed-bed tube reactors as fixed-bed reactors.

In the above-described oxirane synthesis which is preferably employed, particular preference is given to using an isothermal fixed-bed reactor as reactor for step (i) and an adiabatic fixed-bed reactor for step (iii) with the hydroperoxide being separated off in a separation apparatus in step (ii).

The oxiranes used for the process of the present invention are thus preferably prepared in an isothermal fixed-bed reactor and an adiabatic fixed-bed reactor.

It is also possible to react a plurality of organic compounds with the hydroperoxide. It is likewise conceivable to use a plurality of hydroperoxides or solvents for the reaction. If, for example, two solvents are used, they can be separated successfully by the process of the present invention by distillation in a dividing wall column having two side offtakes for liquid, as long as the boiling points are not too close together.

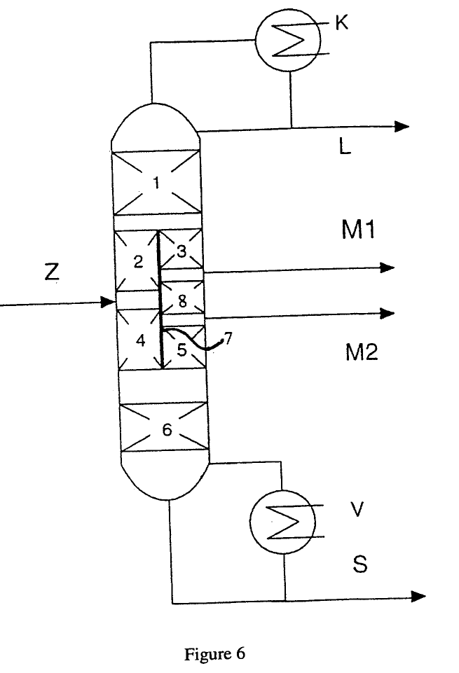

A dividing wall column having two side offtakes is shown schematically in FIG. 6. Here, the lower-boiling solvent is taken off at the upper side offtake M1 and the higher-boiling solvent is taken off at the lower side offtake M2. In this arrangement, the region of thermal coupling 8 preferably has from five to fifty percent, more preferably from fifteen to thirty percent, of the total number of theoretical plates in the column.

In a preferred embodiment of the oxirane synthesis, hydrogen peroxide is used as hydroperoxide and the organic compound is brought into contact with a heterogeneous catalyst during the reaction. Further preference is then given to propylene being used as organic compound and the oxirane being propylene oxide. It is also preferred that the reaction be carried out in methanol as solvent.

A particularly preferred embodiment of the process of the present invention thus provides for the continuously operated distillation in a dividing wall column of the methanol used as solvent in the coproduct-free synthesis of propylene oxide.

The invention also provides an apparatus for carrying out a continuously operated process for the distillation of the solvent used in the synthesis of an oxirane by reaction of a hydroperoxide with an organic compound, which comprises at least one reactor for preparing the oxirane and at least one dividing wall column having one or two more side offtakes for the distillation of the solvent, with the dividing wall column also being able to be in the form of thermally coupled columns.

In a specific embodiment of the apparatus for carrying out a continuously operated process for the distillation of the solvent used in the synthesis of an oxirane by reaction of a hydroperoxide with an organic compound, the apparatus comprises at least one isothermal reactor and one adiabatic reactor for preparing the oxirane in steps (i) and (iii) and a separation apparatus for separating off the hydroperoxide in step (ii) and a dividing wall column or two thermally coupled columns for the distillation of the solvent.

The invention is illustrated by the following example.

### EXAMPLE

Propylene oxide was prepared from propylene by reaction with hydrogen peroxide using the process described in WO 00/07965. The solvent mixture obtained had the following approximate composition:
about 0.2% by weight of low-boiling components including the key components acetaldehyde, methyl formate,
about 80% by weight of methanol, and
about 18.8% by weight of high-boiling components including the key components water, methoxypropanols, 1,2-propylene glycol.

The objective was to limit the sum of the impurities in the methanol to not more than 5% by weight by purifying distillation. For this purpose, the mixture was distilled with the aid of a dividing wall column having a side offtake, with the desired material being taken off from the side offtake of the column. The heating power of the bottom vaporizer was set so that the total concentration of the key components in the product taken off at the side was less than 5% by weight.

The required energy content of the distillation was used as a measure of the effectiveness of the separation. As column configurations, the arrangements shown in the table were selected:

| | | Energy |
|---|---|---|
| | Energy requirement/(kg/h) | saving |
| Column configuration | [kW/(kg/h)] | [%] |
| Conventional column | 0.68 | — |
| with side offtake | | |
| Two conventional | 0.58 | 14.7 |
| columns connected in | | |
| series | | |
| Dividing wall column | 0.45 | 33.8 |

It is clear that the dividing wall configuration had a considerable energy advantage over the two conventional distillation arrangements, since the energy consumption required for the distillation was significantly lower than in the distillation using the conventional columns.

The methanol obtained by distillation in the dividing wall column could be reused for the oxirane synthesis.

### LIST OF REFERENCE NUMERALS FOR FIGS. 1 to 6

1 Combined region of the inflow and offtake part of the dividing wall column
2 Enrichment section of the inflow part
3 Stripping section of the offtake part
4 Stripping section of the inflow part
5 Enrichment section of the offtake part
6 Combined region of the inflow and offtake part
7 Dividing wall
8 Region of thermal coupling
Z Feed
L Low boilers
M Side offtake for intermediate boilers
M1 Side offtake for lower-boiling solvent
M2 Side offtake for higher-boiling solvent
S High boilers
K Condenser
V Vaporizer
d Vapor
f Liquid

Horizontal and diagonal or indicated diagonal lines in the columns symbolize packing made up of random packing elements or ordered packing which may be present in the column.

1-10. (canceled)

11. A process for the continuously operated distillation of the solvent used in the synthesis of an oxirane by reaction of a hydroperoxide with an organic compound, wherein the mixture comprising the solvent which is obtained in the synthesis and subsequent work-up is separated in a dividing wall column into a low-boiling fraction, an intermediate-boiling fraction and a high-boiling fraction and the solvent is taken off as intermediate-boiling fraction from the side offtake of the column.

12. The process as claimed in claim 11, wherein the organic compound used is propylene, the oxirane is propylene oxide and the solvent used is methanol.

13. The process as claimed in claim 11, wherein the dividing wall column has from 15 to 60 theoretical plates.

14. The process as claimed in claim 11, wherein the distillation is carried out at a pressure of from 0.5 to 15 bar and a temperature of from 30 to 140° C., with the pressure being measured in the top of the column and the distillation temperature being measured at the side offtake.

15. The process as claimed in claim 12, wherein the dividing wall column has from 15 to 60 theoretical plates and wherein the distillation is carried out at a pressure of from 0.5 to 15 bar and a temperature of from 30 to 140° C., with the pressure being measured in the top of the column and the distillation temperature being measured at the side offtake.

16. The process as claimed in claim 11, wherein the dividing wall column is in the form of two thermally coupled columns.

17. The process as claimed in claim 16, wherein the solvent mixture is separated into the low-boiling, intermediate-boiling and high-boiling fractions in the column located downstream of the feed column, or
the low-boiling and high-boiling fractions are taken off from the solvent mixture in the feed column and the intermediate-boiling fraction is taken off in the downstream column, or
the high-boiling fraction is taken off from the solvent mixture in the feed column and the low-boiling and intermediate-boiling fractions are taken off in the downstream column, or
the low-boiling fraction is taken off from the solvent mixture in the feed column and the intermediate-boiling and high-boiling fractions are taken off in the downstream column.

18. The process as claimed in claim 16, wherein the liquid bottom stream taken from one of the coupled columns is partly or completely vaporized before it is fed to the other column, and the gaseous top stream taken from one of the coupled columns is partly or completely condensed before it is fed to the other column.

19. The process as claimed in claim 16, wherein the liquid bottom stream taken from one of the coupled columns is partly or completely vaporized before it is fed to the other column, or the gaseous top stream taken from one of the coupled columns is partly or completely condensed before it is fed to the other column.

20. The process as claimed in claim 11, wherein the product mixture comprising the oxirane is prepared by a process comprising at least the steps (i) to (iii):
(i) reaction of the hydroperoxide with the organic compound to give a product mixture comprising the reacted organic compound and unreacted hydroperoxide,
(ii) separation of the unreacted hydroperoxide from the mixture resulting from step (i),
(iii) reaction of the hydroperoxide which has been separated off in step (ii) with the organic compound,
with an isothermal fixed-bed reactor being used in step (i), an adiabatic fixed-bed reactor being used in step (iii), a separation apparatus being used in step (ii) and hydrogen peroxide being used as hydroperoxide and the organic compound being brought into contact with a heterogeneous catalyst during the reaction.

21. The process as claimed in claim 20, wherein the organic compound used is propylene, the oxirane is propylene oxide and the solvent used is methanol.

22. The process as claimed in claim 21, wherein the dividing wall column has from 15 to 60 theoretical plates and wherein the distillation is carried out at a pressure of from 0.5 to 15 bar and a temperature of from 30 to 140° C., with the pressure being measured in the top of the column and the distillation temperature being measured at the side offtake.

23. The process as claimed in claim 20, wherein the dividing wall column is in the form of two thermally coupled columns.

24. The process as claimed in claim 23, wherein the solvent mixture is separated into the low-boiling, intermediate-boiling and high-boiling fractions in the column located downstream of the feed column, or
the low-boiling and high-boiling fractions are taken off from the solvent mixture in the feed column and the intermediate-boiling fraction is taken off in the downstream column, or
the high-boiling fraction is taken off from the solvent mixture in the feed column and the low-boiling and intermediate-boiling fractions are taken off in the downstream column, or
the low-boiling fraction is taken off from the solvent mixture in the feed column and the intermediate-boiling and high-boiling fractions are taken off in the downstream column.

25. The process as claimed in claim 24, wherein the liquid bottom stream taken from one of the coupled columns is partly or completely vaporized before it is fed to the other column, and the gaseous top stream taken from one of the coupled columns is partly or completely condensed before it is fed to the other column.

26. The process as claimed in claim 24, wherein the liquid bottom stream taken from one of the coupled columns is partly or completely vaporized before it is fed to the other column, or the gaseous top stream taken from one of the coupled columns is partly or completely condensed before it is fed to the other column.

27. A process for the continuously operated distillation of the methanol solvent used in the synthesis of propylene oxide by reaction of hydrogen peroxide with propene, wherein the mixture comprising methanol which is obtained in the synthesis and subsequent work-up is separated in a dividing wall column having from 15 to 60 theoretical plates into a low-boiling fraction, an intermediate-boiling fraction and a high-boiling fraction and the methanol is taken off as intermediate-boiling fraction from the side offtake of the column, the distillation in the column being carried out at a pressure of from 0.5 to 15 bar and a temperature of from 30 to 140° C., with the pressure being measured in the top of the column and the distillation temperature being measured at the side offtake, which process further comprises at least the steps (i) to (iii)
(i) reaction of hydrogen peroxide with propene to give a product mixture comprising propylene oxide and unreacted hydrogen peroxide,
(ii) separation of the unreacted hydrogen peroxide from the mixture resulting from step (i),
(iii) reaction of the hydrogen peroxide which has been separated off in step (ii) with propene,
with at least one isothermal fixed-bed reactor being used in step (i), one adiabatic fixed-bed reactor being used in step (iii), and a separation apparatus being used in step (ii).

28. An apparatus for carrying out a continuously operated process for the distillation of the solvent used in the synthesis of an oxirane by reaction of a hydroperoxide with an organic compound, which comprises at least one isothermal fixed-bed reactor and one adiabatic fixed-bed reactor and a separation apparatus for preparing an oxirane in a process comprising at least the steps (i) to (iii):
(i) reaction of the hydroperoxide with the organic compound to give a product mixture comprising the reacted organic compound and unreacted hydroperoxide,
(ii) separation of the unreacted hydroperoxide from the mixture resulting from step (i),
(iii) reaction of the hydroperoxide which has been separated off in step (ii) with the organic compound,
with the isothermal fixed-bed reactor being used in step (i), the adiabatic fixed-bed reactor being used in step (iii), and the separation apparatus being used in step (ii), the apparatus further comprising a dividing wall column or two thermally coupled columns for the distillation of the solvent.
